SEQUENCE LISTING (3 sequences)

SEQ ID NO: 1 - DNA, 2304 bp, Geobacillus sp.
  source 1..2304: mol_type = genomic DNA
atgtggaaggttccaaaattcattaaacaatcatatctcgtttttcttcttgctctattgctctattcttcatttggtttttcgttttccaggactgaggcgactacatctacaggagctttaggaccagttaccccgaaagacacaatatatcagatcgtgacggaccgtttttttgatggcgacccatcaaataacaagcctcctggttttgatcctaccctgtttgatgatccggacggcaataaccaggggaacggaaaagatttaaagttgtatcaaggcggtgatttccaaggaatcatagataaaattccttatttaaaaaatatggggataactgccgtttggatttccgctccttatgaaaatagggacactgtgatagaagattatcaatcagatggaagtattaatcgttggaccagtttccacggttaccatgcaagaaattattttgcaactaacaaacattttggtactatgaaagattttattagactacgcgatgctttgcatcaaaatggaattaaactagtgatagattttgtatccaatcattcgagtcgttggcaaaacccgacattgaattttgcgcctgaagatggtaaattatatgaacctgacaaggatgcgaatggcaattacgtatttgatgctaatggagagcctgcggattacaacggtgatggaaaagttgagaatctcctggcggatccacataacgacgtgaatggttttttccatggtctgggtgaccggggcaacgatacttctcgttttggctaccgttacaaagatctaggttctttggctgattattctcaggaaaatgcactagtggttgaacatttggagaaagcagctaaattttggaaatcaaaagggatcgatggttttcgacatgatgccactttgcatatgaatcctgcatttgtgaagggatttaaagatgcaattgattcagatgcaggtggcccggttacccattttggtgaatttttcattggaagaccggatcccaagtatgatgagtaccggacatttcctgaacgaacaggagtcaacaacttggattttgaatatttccgtgcggccacaaacgcatttgggaacttttctgaaacgatgagttcctttggtgatatgatgatcaagacaagtaatgattacatttatgaaaatcaaacagttactttcttggataatcatgatgtaacaagatttcgctatattcaaccaaacgataaaccttatcatgcagctctcgctgtcttgatgacatcacgtgggattcctaacatttattacggaacagagcaatatctgatgccgtcagactcaagtgacattgcgggtcggatgtttatgcagacttctactaacttcgatgaaaataccactgcatataaagtcattcaaaagctttcaaacttaagaaaaaataatgaagctattgcctatggaaccacagaaattttatacagcacaaatgatgtactggtctttaaaagacagttctatgataaacaagtaattgtagcggtaaaccgacaaccggatcaaacgtttaccattccggagttagatacgactcttccagtaggaacctatagtgatgtactgggtggactgttatatgggagttcaatgagcgtaaataatgtcaacggtcaaaacaaaatttctagctttaccttgtctggaggagaggtcaatgtctggtcgtataacccatcattggggactttaactccaaggattggcgacgttatttccaccatgggacgtcccggtaataccgtttacatttacggtactggattaggaggaagcgtaacagtcaaattcggttctactgttgctactgtggtgtcaaacagcgatcaaatgattgaggctatagttccaaacactaatcctggaattcaaaatattacagttacaaaaggatctgtaaccagtgatcctttccgatatgaggtcctatccggcgatcaggtgcaagtaatttttcatgtgaatgccacaacgaattggggggaaaacatttatgttgtcggaaacattccagagttgggaagctgggatccgaaccaatcgtctgaggcgatgttaaatccgaactatccagaatggttcttgccagtgagtgtgcccaagggagctacttttgaattcaagtttatcaaaaaagataacaatggaaatgtcatttgggaaagcaggagcaacagagtatttaccgcaccgaacagttcgaccggtactattgacacccctttatatttttgggataactaa

SEQ ID NO: 2 - protein, 767 aa, Geobacillus sp.
MWKVPKFIKQSYLVFLLALLLYSSFGFSFSRTEATTSTGALGPVTPKDTIYQIVTDRFFDGDPSNNKPPGFDPTLFDDPDGNNQGNGKDLKLYQGGDFQGIIDKIPYLKNMGITAVWISAPYENRDTVIEDYQSDGSINRWTSFHGYHARNYFATNKHFGTMKDFIRLRDALHQNGIKLVIDFVSNHSSRWQNPTLNFAPEDGKLYEPDKDANGNYVFDANGEPADYNGDGKVENLLADPHNDVNGFFHGLGDRGNDTSRFGYRYKDLGSLADYSQENALVVEHLEKAAKFWKSKGIDGFRHDATLHMNPAFVKGFKDAIDSDAGGPVTHFGEFFIGRPDPKYDEYRTFPERTGVNNLDFEYFRAATNAFGNFSETMSSFGDMMIKTSNDYIYENQTVTFLDNHDVTRFRYIQPNDKPYHAALAVLMTSRGIPNIYYGTEQYLMPSDSSDIAGRMFMQTSTNFDENTTAYKVIQKLSNLRKNNEAIAYGTTEILYSTNDVLVFKRQFYDKQVIVAVNRQPDQTFTIPELDTTLPVGTYSDVLGGLLYGSSMSVNNVNGQNKISSFTLSGGEVNVWSYNPSLGTLTPRIGDVISTMGRPGNTVYIYGTGLGGSVTVKFGSTVATVVSNSDQMIEAIVPNTNPGIQNITVTKGSVTSDPFRYEVLSGDQVQVIFHVNATTNWGENIYVVGNIPELGSWDPNQSSEAMLNPNYPEWFLPVSVPKGATFEFKFIKKDNNGNVIWESRSNRVFTAPNSSTGTIDTPLYFWDN

SEQ ID NO: 3 - protein, 733 aa, Geobacillus sp.
TTSTGALGPVTPKDTIYQIVTDRFFDGDPSNNKPPGFDPTLFDDPDGNNQGNGKDLKLYQGGDFQGIIDKIPYLKNMGITAVWISAPYENRDTVIEDYQSDGSINRWTSFHGYHARNYFATNKHFGTMKDFIRLRDALHQNGIKLVIDFVSNHSSRWQNPTLNFAPEDGKLYEPDKDANGNYVFDANGEPADYNGDGKVENLLADPHNDVNGFFHGLGDRGNDTSRFGYRYKDLGSLADYSQENALVVEHLEKAAKFWKSKGIDGFRHDATLHMNPAFVKGFKDAIDSDAGGPVTHFGEFFIGRPDPKYDEYRTFPERTGVNNLDFEYFRAATNAFGNFSETMSSFGDMMIKTSNDYIYENQTVTFLDNHDVTRFRYIQPNDKPYHAALAVLMTSRGIPNIYYGTEQYLMPSDSSDIAGRMFMQTSTNFDENTTAYKVIQKLSNLRKNNEAIAYGTTEILYSTNDVLVFKRQFYDKQVIVAVNRQPDQTFTIPELDTTLPVGTYSDVLGGLLYGSSMSVNNVNGQNKISSFTLSGGEVNVWSYNPSLGTLTPRIGDVISTMGRPGNTVYIYGTGLGGSVTVKFGSTVATVVSNSDQMIEAIVPNTNPGIQNITVTKGSVTSDPFRYEVLSGDQVQVIFHVNATTNWGENIYVVGNIPELGSWDPNQSSEAMLNPNYPEWFLPVSVPKGATFEFKFIKKDNNGNVIWESRSNRVFTAPNSSTGTIDTPLYFWDN

(19) United States
(12) Patent Application Publication
(10) Pub. No.: US 2025/0101478 A1
(43) Pub. Date: Mar. 27, 2025

(54) USE OF MALTOTRIOSYL TRANSFERASE

(71) Applicant: AMANO ENZYME INC., Nagoya-shi (JP)

(72) Inventors: Masamichi Okada, Kakamigahara-shi (JP); Shotaro Yamaguchi, Kakamigahara-shi (JP)

(73) Assignee: AMANO ENZYME INC., Nagoya-shi (JP)

(21) Appl. No.: 18/829,521

(22) Filed: Sep. 10, 2024

Related U.S. Application Data
(63) Continuation of application No. 15/063,962, filed on Mar. 8, 2016, now abandoned.
Application No. 15/063,962 is a continuation-in-part of application No. 13/982,923, filed on Oct. 4, 2013, now abandoned.

(30) Foreign Application Priority Data
Feb. 4, 2011 (JP) 2011-023281
Jul. 26, 2011 (JP) 2011-162973

(51) Int. Cl. C12P 19/04; A23L 33/21; C12C 5/00; C12C 7/047; C12C 11/00; C12C 12/00; C12P 19/18

(52) U.S. Cl. CPC C12P 19/04; A23L 33/21; C12C 5/004; C12C 7/047; C12C 11/00; C12C 12/00; C12C 12/002; C12P 19/18

(57) ABSTRACT

The present invention is intended to provide a novel use of a maltotriosyl transferase. The present invention provides a method for producing rice cakes or noodles, including a step of heat-treating a dough containing maltotriosyl transferase thereby gelatinizing starch in the dough. The present invention also provides a method for producing an indigestible saccharide, including a step of allowing maltotriosyl transferase to act on a saccharide.

### CROSS REFERENCE TO RELATED APPLICATIONS

This application is a continuation of U.S. application Ser. No. 15/063,962, filed on Mar. 8, 2016, which is a continuation-in-part of U.S. application Ser. No. 13/982,923, filed on Oct. 4, 2013, which is a National Stage Entry of PCT/JP2012/052082, filed on Jan. 31, 2012, which claims priority to Japanese application No. 2011-023281, filed on Feb. 4, 2011, and Japanese application No. 2011-162973, filed on Jul. 26, 2011, all of which are hereby incorporated by reference in their entireties.

### SEQUENCE LISTING

The instant application contains a Sequence Listing which has been submitted in.txt format and is hereby incorporated by reference in its entirety. The.txt Sequence Listing file is named 1416865.215US43-Sequence Listing.xml and was created on Nov. 19, 2024 and is 7,394 bytes in size.

### FIELD OF THE INVENTION

The present invention relates to a novel use of maltotriosyl transferase, and specifically to a method for producing rice cakes, a method for producing noodles, and a method for producing an indigestible saccharide, and a method for producing a glycoside using maltotriosyl transferase.

### BACKGROUND OF THE INVENTION

Known examples of industrially used glycosyltransferases include α-glucosidase (production of isomaltooligosaccharide or nigerooligosaccharide), β-fructofuranosidase (production of fructooligosaccharide or lactosucrose), β-galactosidase (production of galactooligosaccharide), α-glucosyltransferase (production of palatinose), cyclodextrin glucanotransferase (production of cyclodextrin or coupling sugar), and branching enzymes (production of highly branched cyclic dextrin). Alfa-glucosidase and branching enzymes act on polysaccharides and oligosaccharides containing α-1,4 bonds to catalyze transglucosylation. Alfa-glucosidase catalyzes transglucosylation of monosaccharides, and branching enzymes catalyze transglucosylation of oligosaccharides of four or more sugars or polysaccharides.

One of uses of enzymes is the production of processed food containing starch. Starch aging causes considerable problems such as the deterioration of storage stability. Starch aging is mostly caused by aging of amylose molecules contained in starch, more specifically association and insolubilization of the amylose molecules (Non-patent Document 1). Therefore, control of starch aging through depolymerization of starch was studied, which allowed control of starch aging to some degree. However, depolymerization impairs the original properties of starch. Furthermore, under this method, decomposed starch has increased reducing power, and is stained upon reaction with protein or amino acid under heating. Therefore, this method has limited uses (Patent Document 1). Accordingly, aging control without depolymerization of starch has been studied.

Under the above circumstances, the present applicant reported a novel glycosyltransferase which catalyzes the transglucosylation of maltotriose units (hereinafter referred to as “maltotriosyl transferase” or “the present enzyme”) and a method for producing the same in a preceding patent application, and showed the examples of the uses. (Patent Document 2).

### PRIOR ART DOCUMENTS

### Patent Document

Patent Document 1: Japanese Unexamined Patent Application Publication No. 2001-294601

Patent Document 2: WO 2011/001722

Patent Document 3: Japanese Unexamined Patent Application Publication No. 54-49354

Patent Document 4: Japanese Unexamined Patent Application Publication No. 2008-194024

Patent Document 5: Japanese Unexamined Patent Application Publication No. 9-107900

Patent Document 6: WO 2008/136331

### Non-Patent Document

Non-patent Document 1: Okada et Al., Denpun Kagaku (Journal of the Japanese Society of Starch Science), 30 (2), p. 223-230 (1983)

### SUMMARY OF THE INVENTION

The present invention is intended to provide a novel use of maltotriosyl transferase found by the inventors.

In consideration of the above-described problems, the inventors carried out various investigations, and focused attention on the production and processing of rice cakes, and production of indigestible saccharide. The results of experiments indicated that the present enzyme has unexpectedly high heat resistance, sufficiently acts even when added before steaming the rice cake dough, and effectively prevents aging of rice cake. This fact means that the present enzyme dispenses with the step of adding an enzyme after steaming the rice cake dough (which requires the temperature control process for moderately cooling the rice cake dough before adding the enzyme), and allows the production of rice cakes having high aging resistance through a more simple production process. Also in the method for producing noodles, the same effect as the method for producing rice cakes is obtained by subjecting the dough (noodle dough) containing the present enzyme to heat treatment for gelatinizing the starch in the dough, in the same manner as in the method for producing rice cakes. In addition, as a result of the study by the inventors, the present enzyme is useful for the production of indigestible saccharides, particularly because it allows the production on an industrial scale.

Rice cakes have a unique texture (chewy texture), but the texture is impaired as the starch ages with the lapse of time. Various measures are taken for preventing the deterioration of rice cake quality. Many of the measures use enzymes such as β amylase (for example, Patent Document 3) and transglucosidase (for example, Patent Document 4). When an enzyme is used, in consideration of action behavior and deactivation of the enzyme, the enzyme is usually added after moderately cooling the steamed rice cake dough (for example, Patent Document 5). However, such production method is complicated because it requires the step of cooling the rice cake dough after steaming (which requires temperature control, and may require the step of allowing the enzyme to react after adding the enzyme). The preceding patent application by the present applicant (Patent Document 2) describes the production process of rice cake using rice flour (Joshinko) as an example of the use of the present enzyme. In the process, the enzyme is added when the temperature of the dough becomes about 65° C., and thus the similar problem as above is posed. On the other hand, according to the new production method established by the present study, a rice cake dough containing an enzyme is subjected to heat treatment such as steaming. Therefore, the method dispenses with the above-described prior art steps carried out after steaming, and markedly improves workability. In addition, the production method of the present invention may use ingredients which have been mixed with the enzyme (premix powder), thereby further improves the workability.

Indigestible saccharides are also found to be useful, and various methods for producing them are developed. For example, Patent Document 4 describes the method for producing an indigestible saccharide using α-glucosyl transferase. In the production method, the temperature during reaction of the enzyme is low (40° C.) likely due to the properties of the enzyme. The reaction at such a relatively low temperature involves high risks of microorganism contamination, and thus is not suitable to the production on industrial scale. On the other hand, the new production method established by the present study may be used under high temperature conditions because it uses an enzyme having high heat resistance, and thus reduces the risk of microorganism contamination. Accordingly, the method is also suitable to the production of indigestible saccharides on industrial scale.

As a result of further study on indigestible saccharides, it was found that the present enzyme is effective for the increase of the indigestible saccharide content in beer or a beer-like beverage. It was also found that the present enzyme is markedly effective for the production of hetero-oligosaccharides and glycosides. In addition, it was shown that the use of the present enzyme in the production of noodles achieves the same effect as that achieved in the production of rice cakes.

The present invention described below is, as is evident from the above explanations, a solution to prior art, and brings about marked advantages.
[1] A method for producing an indigestible saccharide, comprising a step of allowing maltotriosyl transferase to act on a dextrin, wherein the maltotriosyl transferase is composed of an amino acid sequence SEQ ID NO. 3, an amino acid sequence which is 70% or more identical to the amino acid sequence SEQ ID NO. 3, or a fragment of the amino acid sequence SEQ ID NO. 3 showing maltotriosyl transferase activity.
[2] The method according to [1], wherein the step is carried out using one or more enzymes selected from the group consisting of cyclodextrin glucanotransferase, a-amylase (only those having transglycosylation activity), pullulanase, and isoamylase in combination with the maltotriosyl transferase.
[3] The production method according to [1], wherein the reaction temperature during the step is from 50° C. to 70° C.
[4] The method for producing an indigestible saccharide according to [1], wherein the step is carried out in the presence of a receptor substrate, and a hetero-oligosaccharide is formed as the indigestible saccharide.
[5] The method for producing an indigestible saccharide according to [4], wherein the receptor substrate is monosaccharide and/or sugar alcohol.
[6] The method for producing an indigestible saccharide according to [4], wherein the receptor substrate is one or more sugars or sugar alcohols selected from the group consisting of D-xylose, D-fructose, D-glucose, L-fucose, L-sorbose, Dmannose, D-sorbitol, D-galactose, N-acetyl glucosamine, L-arabinose, D-ribose, and Lrhamnose.
[7] The method for producing an indigestible saccharide according to [1], wherein the dextrin is formed by saccharification of a saccharide ingredient used for producing beer or a beer-like beverage.
[8] The method for producing an indigestible saccharide according to [7], wherein the saccharide ingredient is one or more ingredients selected from the group consisting of malt, barley, wheat, rye, oat, corn, rice, kaoliang, potato, soybean, pea bean, chick-pea, and corn starch.
[9] The production method according to [1], wherein the maltotriosyl transferase has the following enzymatic properties:
the enzyme acts on polysaccharides and oligosaccharides having a-1,4 glucoside bonds to transfer maltotriose units to saccharides; when the enzyme acts on maltotetraose as the substrate, the ratio between the production rates of maltoheptaose and maltotriose is from 9:1 to 10:0 over the whole range of the substrate concentration from 0.67% (w/v) to 70% (w/v).
[10] The production method according to [1], wherein the maltotriosyl transferase is an enzyme derived from a microorganism.
[11] The production method according to [1], wherein the maltotriosyl transferase is an enzyme derived from Geobacillus species.
[12] The production method according to [11], wherein the Geobacillus microorganism is Geobacillus sp. APC9669 (accession number NITE BP-770).
[13] The production method according to [1], wherein the maltotriosyl transferase has the following enzymatic properties:
(1) action: the enzyme acts on polysaccharides and oligosaccharides having a-1,4 glucoside bonds to transfer maltotriose units to saccharides;
(2) substrate specificity: the enzyme acts on soluble starch, amylose, amylopectin, maltotetraose, maltopentaose, and maltohexaose, but does not act on a-cyclodextrin, b-cyclodextrin, g-cyclodextrin, maltotriose, and maltose; and
(3) molecular weight: about 83,000 (by SDS-PAGE).
[14] A composition containing an indigestible saccharide produced by the production method according to [1].
[15] The composition according to [14], which is a pharmaceutical composition, a quasi-drug composition, or a food composition.
[16] The composition according to [15], wherein the food composition is beer or a beer-like beverage.
[17] The method for producing an indigestible saccharide according to [1], wherein the maltotriosyl transferase has the following enzymatic properties:
the enzyme acts on polysaccharides and oligosaccharides having a-1,4 glucoside bonds to transfer maltotriose units to saccharides; when the enzyme acts on maltotetraose as the substrate, the ratio between the production rates of maltoheptaose and maltotriose is from 9:1 to 10:0 over the whole range of the substrate concentration from 0.67% (w/v) to 70% (w/v).
[18] A method for producing a dextrin solution which is hard to retrograde, comprising a step of allowing maltotriosyl transferase to act on dextrin in a solution.
[19] A dextrin solution produced by the production method according to [18].
[20] A composition comprising the dextrin solution according to [19].

### BRIEF DESCRIPTION OF THE DRAWINGS

FIG. 1 is a graph showing the optimal temperature of the maltotriosyl transferase derived from Geobacillus sp. APC9669.

FIG. 2 is a graph showing the optimal pH of the maltotriosyl transferase derived from Geobacillus sp. APC9669.

FIG. 3 is a graph showing the thermostability of the maltotriosyl transferase derived from Geobacillus sp. APC9669.

FIG. 4 is a graph showing the pH stability of the maltotriosyl transferase derived from Geobacillus sp. APC9669.

FIG. 5 shows the result of SDS-PAGE of the maltotriosyl transferase. Lane 1: molecular weight marker, and lane 2: maltotriosyl transferase.

FIG. 6 shows the anti-retrograding effect of maltotriosyl transferase on dextrin solution.

### DETAILED DESCRIPTION OF THE INVENTION

### (Term)

In the present invention, the term “DNA coding a protein” refers to the DNA which gives the protein upon expression, more specifically the DNA having a base sequence corresponding to the amino acid sequence of the protein. Accordingly, codon degeneracy is taken into consideration.

In the present description, the term “isolated” may be replaced with “purified”. When the enzyme of the present invention (maltotriosyl transferase) is derived from a natural material, the term “isolated” used for the enzyme means that the enzyme is substantially free of components of the natural material other than the enzyme (specifically substantially free of contaminant protein). Specifically, for example, in the isolated enzyme of the present invention, the content of contaminant proteins is less than about 20%, preferably less than about 10%, more preferably less than about 5%, and even more preferably less than about 1% in the weight equivalence. On the other hand, when the enzyme of the present invention is prepared by a genetic engineering technique, the term “isolated” means that the enzyme is substantially free of other components derived from the host cells or culture solution used. Specifically, for example, in the isolated enzyme of the present invention, the content of contaminant components is less than about 20%, preferably less than about 10%, more preferably less than about 5%, and even more preferably less than about 1% in the weight equivalence. In the present description, the simple term “maltotriosyl transferase” means “isolated maltotriosyl transferase”, unless it is evident that the term has a different meaning. The same applies to the term “the present enzyme” used in place of maltotriosyl transferase.

### Use of Maltotriosyl Transferase 1: Method for Producing Rice Cakes or Noodles

A first aspect of the present invention is to provide a method for producing rice cakes or noodles as a use of maltotriosyl transferase. The present invention preferably uses the maltotriosyl transferase (the present enzyme) disclosed by the inventors in the preceding patent application (see Patent Document 2 for more detail). As described in the preceding patent application, Geobacillus sp. APC9669 was found to produce maltotriosyl transferase, and the enzymatic properties were determined. The enzymatic properties thus determined are listed below.

### (1) Action

The present enzyme is a maltotriosyl transferase, and acts on polysaccharides and oligosaccharides having α-1,4 glucoside bonds as a binding mode to transfer maltotriose units to saccharides.

### (2) Substrate Specificity

The present enzyme favorably acts on soluble starch, amylose, amylopectin, maltotetraose, maltopentaose, and maltohexaose. On the other hand, the present enzyme does not act on α-cyclodextrin, β-cyclodextrin, γ-cyclodextrin, maltotriose, and maltose.

### (3) Molecular Weight

The molecular weight of the present enzyme is about 83,000 (by SDS-PAGE).

### (4) Optimum Temperature

The optimum temperature for the present enzyme is about 50° C. The present enzyme exhibits high activity in the temperature range of about 45° C. to 55° C. The optimum temperature was calculated by the below-described method for measuring maltotriosyl transferase activity (in 10 mmol/L MES buffer solution (pH 6.5)).

### (5) Optimum pH

The optimum pH for the present enzyme is about 7.5. The present enzyme exhibits high activity in the pH range of about 6.5 to 8.0. The optimum pH is determined based on, for example, the measurement in a universal buffer solution.

### (6) Thermostability

The present enzyme exhibits stable activity at 65° C. or lower. The present enzyme keeps 90% or higher level of activity even after treatment for 30 minutes at 65° C. in a 10 mmol/L MES buffer solution (pH 6.5).

### (7) pH Stability

The present enzyme exhibits stable activity in a wide pH range of 5.0 to 10.0. More specifically, the enzyme keeps 85% or higher level of activity after treatment for 30 minutes at 40° C., as long as the pH of the enzyme solution used for the treatment is within the above range.

(8) Isoelectric point

The isoelectric point of the present enzyme is about 4.5 (by Ampholine electrophoresis).

As shown in the prior patent application, when the maltotriosyl transferase produced by Geobacillus sp. APC9669 acts on maltotetraose as substrate, it gives a ratio between the maltoheptaose production rate and maltotriose production rate of 9:1 to 10:0 at any substrate concentration ranging from 0.67 to 70% (w/v), wherein maltoheptaose is a transglycosylation product and maltotriose is a decomposition product, respectively. In other words, the rate of transglucosylation is far higher over the wide substrate concentration range, and the maltoheptaose production rate was 90% or more, taking the sum of the maltoheptaose and maltotriose production rates as 100%.

The rates were compared based on the molar ratios of the products.

The present enzyme is preferably a maltotriosyl transferase derived from Geobacillus sp. APC9669. The term “maltotriosyl transferase derived from Geobacillus sp. APC9669” in this case means a maltotriosyl transferase produced by Geobacillus sp. APC9669 (wild strain or mutant strain), or a maltotriosyl transferase obtained by a genetic engineering technique using the maltotriosyl transferase gene of Geobacillus sp. APC9669 (wild strain or mutant strain). Accordingly, “maltotriosyl transferase derived from Geobacillus sp. APC9669” includes the recombinants produced by host microorganisms into which the maltotriosyl transferase gene (or the modified version of the gene) obtained from Geobacillus sp. APC9669, “Geobacillus sp. APC9669 has been introduced.
NITEBiotechnologyDevelopmentCenterChiba For convenience of explanation, Geobacillus sp. APC9669 which is the source of the present enzyme is referred herein the bacterium producing the present enzyme. The APC9669 strain is deposited on the below-described depository, and is readily available therefrom.
NITEBiotechnologyDevelopmentCenterChiba Depository institution: Patent Microorganisms Depository, (2-5-8, Kazusakamatari, Kisarazu-shi,, 292-0818, Japan)
NITEBiotechnologyDevelopmentCenterChiba Date of deposit (date of receipt): Jun. 2, 2009
NITEBiotechnologyDevelopmentCenterChiba Accession number: NITE BP-770
NITEBiotechnologyDevelopmentCenterChiba

NITEBiotechnologyDevelopmentCenterChiba After culturing the Geobacillus sp. APC9669, the present enzyme is collected from the culture solution and/or bacterial cells. The culture method may be liquid culture or solid culture, and preferably liquid culture. The conditions of liquid culture are described below. NITEBiotechnologyDevelopmentCenterChiba

The medium is not particularly limited as long as it is suitable for growing the microorganism used. Examples of the medium include those containing a carbon source such as glucose, sucrose, genthiobiose, soluble starch, glycerol, dextrin, molasses, or an organic acid, in addition, ammonium sulfate, ammonium carbonate, ammonium phosphate, ammonium acetate, or a nitrogen source such as peptone, yeast extract, corn steep liquor, casein hydrolysate, bran, or meat extract, in addition an inorganic salt such as a potassium salt, a magnesium salt, a sodium salt, a phosphate, a manganese salt, an iron salt, or a zinc salt. In order to accelerate the growth of the microorganism, a vitamin and an amino acid may be added to the medium. The pH of the medium is adjusted to, for example, about 3 to 10, and preferably about 7 to 8, and the incubation temperature is normally from about 10 to 80° C., preferably about 30 to 65° C. The microorganism is cultured for about 1 to 7 days, preferably for about 2 to 4 days under aerobic conditions. The culture method may be, for example, a shake culture method or an aerobic deep culture method using a jar fermenter.

After culturing under the above-described conditions, a maltotriosyl transferase is collected from the culture solution or bacterial cells. When the enzyme is collected from the culture solution, for example, the culture supernatant is subjected to, for example, filtration or centrifugation thereby removing insoluble matter, followed by separation and purification through an appropriate combination of, for example, concentration using an ultrafiltration membrane, salting out by ammonium sulfate precipitation, dialysis, and various chromatography procedures such as ion exchange chromatography, and thus obtaining the present enzyme.

On the other hand, when the enzyme is collected from bacterial cells, the bacterial cells are crushed by, for example, pressurization or ultrasonication, and then subjected to separation and purification in the same manner as described-above, and thus obtaining the present enzyme. Alternatively, the bacterial cells may be collected in advance from the culture solution by, for example, filtration or centrifugation, and then the above-described procedure (crushing of bacterial cells, separation, and purification) may be carried out.

Confirmation of expression and identification of the expression product are readily achieved using an antibody against the maltotriosyl transferase. Alternatively, the expression may be confirmed by measuring the maltotriosyl transferase activity.

The maltotriosyl transferase for the present invention according to one embodiment contains the amino acid sequence set forth in SEQ ID NO: 3. This amino acid sequence is the amino acid sequence set forth in SEQ ID NO: 2 excluding the signal peptide portion. The amino acid sequence set forth in SEQ ID NO: 2 was deduced from the base sequence (SEQ ID NO: 1) of the gene obtained by cloning Geobacillus sp. APC9669. In general, when the amino acid sequence of a certain protein is partially modified, the modified protein may have equivalent function to the unmodified protein. More specifically, modification of the amino acid sequence does not substantially influence the function of the protein, and thus the function of the protein may be maintained before and after the modification. Accordingly, for the present invention, use of a protein which is composed of an amino acid sequence equivalent to the amino acid sequence set forth in SEQ ID NO: 3, and has maltotriosyl transferase activity (hereinafter, also referred to as “equivalent protein”) is also contemplated. The term “equivalent amino acid sequence” in this case means an amino acid sequence which is partially different from the amino acid sequence set forth in SEQ ID NO: 3, but the difference does not substantially influence the function of the protein (maltotriosyl transferase activity). The term “maltotriosyl transferase activity” means the activity for polysaccharides and oligosaccharides having α-1,4 glucoside bonds as a binding mode to transfer the maltotriose units to saccharides. The degree of the activity is not particularly limited as long as the function of a maltotriosyl transferase can be exhibited, but is preferably equivalent to or higher than that of the protein composed of the amino acid sequence set forth in SEQ ID NO: 3.

The term “partial difference in the amino acid sequence” typically means mutation (change) in the amino acid sequence caused by deletion or substitution of one to several (up to, for example, 3, 5, 7, or 10) amino acids composing the amino acid sequence, or addition, insertion, or combination thereof of one to several (up to, for example, 3, 5, 7, or 10) amino acids. The difference in the amino acid sequence is acceptable as long as the maltotriosyl transferase activity is maintained (the activity may be varied to a degree). As long as the conditions are satisfied, the position of the difference in the amino acid sequence is not particularly limited, and the difference may arise in a plurality of positions. The term “plurality” means, for example, a number corresponding to less than about 30%, preferably less than about 20%, more preferably less than about 10%, even more preferably less than about 5% of the total amino acids, and most preferably less than about 1%. More specifically, the equivalent protein has, for example, about 70% or more, preferably about 80% or more, even more preferably about 90% or more, even more preferably about 95% or more, and most preferably about 99% or more identity with the amino acid sequence set forth in SEQ ID NO: 3.

Preferably, the equivalence protein is obtained by causing conservative amino acid substitution in an amino acid residue which is not essential for maltotriosyl transferase activity. The term “conservative amino acid substitution” means the substitution of an amino acid residue with another amino acid residue having a side chain with similar properties. Amino acid residues are classified into several families according to their side chains, such as basic side chains (for example, ricin, arginine, and histidine), acidic side chains (for example, aspartic acid and glutamic acid), uncharged polar side chains (for example, glycine, asparagine, glutamine, serine, threonine, tyrosine, and cysteine), nonpolar side chains (for example, alanine, valine, leucine, isoleucine, proline, phenylalanine, methionine, and tryptophan), β branched side chains (for example, threonine, valine, and isoleucine), and aromatic side chains (for example, tyrosine, phenylalanine, tryptophan, and histidine). Conservative amino acid substitution is preferably the substitution between amino acid residues in one family.

The “equivalent protein” may have additional properties. For example, the equivalent protein may have higher stability than the protein composed of the amino acid sequence set forth in SEQ ID NO: 3, may perform a different function performed only at low temperatures and/or high temperatures, or may have a different optimum pH.

The identity (%) between the two amino acid sequences may be determined by, for example, the following procedure. Firstly, the two sequences are aligned for optimal comparison (for example, a gap may be introduced into the first sequence thereby optimizing the alignment with the second sequence). When the molecule at the specific position in the first sequence (amino acid residue) is the same as the molecule at the corresponding position in the second sequence, the molecules at the positions are regarded as identical. The sequence identity is the function of the number of the identical positions common to the two sequences (more specifically, identity (%)=number of identical positions/total number of positions×100), and preferably the number and size of the gaps required for the optimization of the alignment are taken into consideration. Comparison of the two sequences and determination of the identity are achieved using a mathematic algorithm. Specific examples of the mathematic algorithm usable for the comparison of the sequences include, but not limited to, the algorithm described in Karlin and Altschul (1990) Proc. Natl. Acad. Sci. USA 87:2264-68, and modified in Karlin and Altschul (1993) Proc. Natl. Acad. Sci. USA 90:5873-77. These algorithms are incorporated into NBLAST Program and XBLAST Program (version 2.0) described in Altschul et al. (1990) J. Mol. Biol. 215:403-10. For example, under XBLAST Program, when BLAST polypeptide is searched under conditions that score=50 and wordlength=3, an amino acid sequence with a high identity can be obtained. Gapped BLAST described in Altschul et al. (1997), Amino Acids Research 25 (17): 3389-3402 can be used for obtaining a gap alignment for comparison. When BLAST and Gapped BLAST are used, default parameters of corresponding programs (for example, XBLAST and NBLAST) may be used. For more detailed information, see, for example, the website of NCBI. Other examples of the mathematic algorithm usable for the comparison of the sequences include the algorithm described in Myersand Miller (1988) Comput Appl BioSci. 4:11-17. These algorithms are incorporated into, for example, ALIGN Program usable in GENESTREAM Network Server (IGH Montpellier, France) or ISREC Server. When ALIGN program is used for the comparison of amino acid sequences, for example, a PAM120 weight residue table is used, wherein the gap length penalty is 12, and the gap penalty is 4. The identity between the two amino acid sequences is determined according to GAP Program of GCG software package using Blossom 62 matrix or PAM250 matrix, wherein the gap weight is 12, 10, 8, 6, or 4, and the gap length weight is=2, 3, or 4.

The present enzyme may be a portion of a larger protein (for example, a fused protein). Examples of the sequence added to a fused protein include the sequences useful for purification of multiple histidine residues, and addition sequences which ensures stability in recombination production.

The present enzyme having the above-described amino acid sequence is readily prepared by a genetic engineering technique. For example, an appropriate host cell (for example, Escherichia coli) is transformed by a DNA coding the present enzyme (specific example is a DNA having nucleotide sequence of SEQ ID NO:1), and the protein expressed in the transformant is collected, and thereby preparing the present enzyme. The collected protein is treated as appropriate according to the intended use.

The present enzyme thus obtained as a recombinant protein may be subjected to various modifications. For example, the present enzyme composed of a recombinant protein linked to any peptide or protein can be obtained by producing a recombinant protein using a vector into which a DNA coding the present enzyme has been inserted together with other appropriate DNA. In addition, modification for causing addition of a sugar chain and/or a lipid, or N-or C-terminal processing may be carried out. These modifications allow, for example, extraction of a recombinant protein, simplification of purification, or addition of biological functions.

In the method for producing rice cakes according to the present invention, a rice cake dough containing the present enzyme is subjected to heat treatment, thereby gelatinizing starch in the rice cake dough. More specifically, different from prior art production method wherein an enzyme is added after steaming or boiling a rice cake dough, in the present invention, a rice cake dough containing an enzyme is subjected to steaming or boiling. The production method of the present invention including this step does not require the complicated step involving temperature control, in which an enzyme is added after cooling to the predetermined temperature after steaming or boiling, which brings about marked improvement of workability. In prior art method, the enzyme must be added after cooling the rice cake dough. Therefore, it is difficult to uniformly mix the enzyme with the rice cake dough having low flowability, and the dough may have uneven softness. In order to achieve uniform softness, the enzyme must be thoroughly mixed with the whole of the rice cake dough. These problems will not occur under the preferred embodiment of the invention which includes heat treatment using a rice cake dough which has been mixed with an enzyme (details will be described below). In the production method of the present invention, the rice cake dough containing the present enzyme is heated to 80° C. or more, because the dough is heated after adding the enzyme. In other words, at least a portion of the present enzyme is exposed to heat of 80° C. or higher. In this point, the production method of the present invention is evidently different from the prior art method including the addition of an enzyme after cooling the rice cake dough to about 60° C. (the enzyme added will not be exposed to 80° C. or higher temperatures).

Also, in the method for producing noodles according to the present invention, heat treatment is carried out in the same manner as in the method for producing rice cakes, thereby gelatinizing starch in the dough (noodle dough) containing the present enzyme. Through this step, the same effect as the method for producing rice cakes is achieved.

The term “rice cakes” is the generic name of mochi (rice cake), mochi sweets, and mochi dumplings. Examples of mochi include various ones such as Noshi-mochi, Maru-mochi, and Kagami-mochi. Examples of “rice cake sweets” include Gyuhi-mochi, Habutae-mochi, Daifuku-mochi, Ankoro-mochi, Abekawa-mochi, Kusa-mochi, Kashiwa-mochi, Sakura-mochi, Suama, and Yubeshi. Examples of “dumpling” include Mitarashi dumpling, Kusa dumpling, and Shiratama dumpling.

“Rice cake dough” is prepared by adding water and other optional ingredients to ground or floured fresh or processed (for example, immersed, dried, or heat-treated) glutinous rice flour or rice flour (for example, Joshinko, Mochiko, Shiratamako, Dangoko, and Domyojiko), followed by mixing or kneading. The dough may be shaped after mixing or kneading. According to one embodiment of the present invention, the rice cake dough contains a pH controlling agent. The rice cake dough is usually subjected to heat treatment, and then rolling, molding, and other steps to produce a rice cake product.

“Noodle dough” is prepared by adding water and other optional ingredients to ground or floured fresh or processed (for example, immersed, dried, or heat-treated) glutinous rice, wheat, foxtail millet, Japanese millet, common millet, buckwheat, aroid, sweet potato, potato, common sorghum, corn, green bean, or soybean, followed by mixing or kneading. The dough may be shaped after mixing or kneading.

The time of adding the present enzyme is not particularly limited as long as the dough (rice cake dough or noodle dough) containing the present enzyme is subjected to heat treatment. For example, the present enzyme is added during kneading other ingredients before the heat treatment. This method does not require the step of adding the enzyme, and thus improves workability and reduces the production time. Alternatively, the present enzyme may be mixed with one or more of other ingredients in advance (use of a premix powder). Under this method, ingredients which have been mixed with the enzyme (premix powder) are subjected to kneading and subsequent heat treatment and other steps, so that the production method offers improved workability. In these two examples, the dough which has been mixed with the present enzyme is subjected to heat treatment. Alternatively, the other ingredients are kneaded together to make a dough, and then the present enzyme may be added during heat treatment of the dough (more specifically, before initiation, immediately after initiation, or during heat treatment).

The loading of the present enzyme is not particularly limited as long as the intended effect (aging prevention) will be achieved, and may be from 1 U to 20 U for 1 g of the main ingredient. The main ingredient means the ingredient or material providing the base of the dough (rice cake dough or noodle dough). For example, the main ingredient of Shiratama Dango (dumpling) is rice flour (glutinous rice flour).

The heat treatment in the present invention may be carried out under the same conditions as in prior art methods, and when the step is achieved by steaming, for example, the dough is steamed with water vapor for 5 minutes to 20 minutes. The treatment time will not be limited to this example, and may be appropriately adjusted in consideration of the components and amount of the dough.

In the production of so-called Tsukimochi (pounded rice cake), aging prevention effect will be achieved also when maltotriosyl transferase is added to glutinous rice before or during steaming.

### Use of Maltotriosyl Transferase 2: Method for Producing Indigestible Saccharide and Uses Therefor

A second aspect of the present invention is to provide a method for producing an indigestible saccharide as a use of maltotriosyl transferase, and also the uses of the indigestible saccharide produced by the production method. The method for producing an indigestible saccharide according to the present invention uses the same maltotriosyl transferase (the present enzyme) as the first aspect, preferably the maltotriosyl transferase disclosed by the present applicant in the preceding patent application (see Patent Document 2 for more details). In more detail, the method for producing an indigestible saccharide according to the present invention includes a step of allowing maltotriosyl transferase (preferably the present enzyme) to act on a saccharide as the substrate.

The “saccharide” to which the enzyme is acted on is a polysaccharide or oligosaccharide (for example, amylose, amylopectin, or any oligosaccharide) having α-1,4 glucoside bonds. The “saccharide” in the present invention may be a mixture of two or more polysaccharides or oligosaccharides. The “saccharide” is preferably a dextrin (starch hydrolysate). Dextrins are usually obtained by the action of α-amylase on starch, or acidolysis of starch. The starch to be the ingredient is not particularly limited. Examples of starch include corn starch, potato starch, wheat starch, rise starch, tapioca starch, and cassava starch. Two or more kinds of starch may be used in combination. At present, corn starch is particularly preferred from the viewpoints of supply and cost. The dextrin may be selected from various kinds of commercially available dextrin (starch hydrolysate).

In the present description, the term “indigestible saccharide” means a saccharide containing an indigestible component. The indigestible component content is not particularly limited. The production method according to the present invention can produce an indigestible saccharide having a relatively high indigestible component content (50% to 60% or more). In the present description, the term “indigestible saccharide” includes the terms “indigestible dextrin”, “indigestible starch”, and “indigestible oligosaccharide”. For example, those produced using a dextrin as the substrate may be referred to as “indigestible dextrin”, focusing on the substrate as an ingredient.

The loading of the present enzyme is not particularly limited, and may be from 1 U to 40 U for 1 g of saccharide.

The temperature during the enzyme reaction is not particularly limited, but preferably from 30° C. to 70° C., thereby allowing the present enzyme to sufficiently act. On the other hand, the temperature is preferably higher for reducing the risk of microorganism contamination, so that the reaction temperature is, for example, from 50° C. to 70° C.

As described in examples, the combination of the present enzyme and cyclodextrin glucanotransferase and/or pullulanase increased the formation of the indigestible component. These two enzymes used in combination do not form indigestible components by themselves, but the combined use with the present enzyme achieved a synergistic effect, and increased the indigestible component. On the basis of this finding, according to one embodiment of the present invention, the present enzyme is used in combination with one or more enzymes selected from the group consisting of cyclodextrin glucanotransferase, α-amylase (only those having transglycosylation activity), pullulanase, and isoamylase, and the combination is allowed to act on a dextrin. Alfa-amylase likely achieves the same effect as cyclodextrin glucanotransferase as long as it has transglycosylation activity, and thus may be used in combination with the present enzyme. Isoamylase shows a common action with pullulanase, and thus may be used in combination with the present enzyme. Examples of the cyclodextrin glucanotransferase include TORUZYME 3.0 L (manufactured by NOVOZYMES), CONTIZYME (manufactured by Amano Enzyme Inc.), and those derived from Geobacillus stearothermophilus. Examples of the α-amylase having transglycosylation activity include the amylase derived from Bacillus circulans (Patent Document 4). Examples of the pullulanase include KLEISTASE PL45 (manufactured by Amano Enzyme Inc.), and Pullulanase “AMANO” 3 (manufactured by Amano Enzyme Inc.). Examples of the isoamylase include the isoamylase derived from Pseudomonas sp. (manufactured by Megazyme), and GODO-FIA (manufactured by GODO SHUSEI Co., Ltd.). The usage of the enzymes used in combination with the present enzyme may be established based on, for example, a preliminary experiment, with reference to the usage shown in the examples. For example, the usage of cyclodextrin glucanotransferase is from 0.1 mg to 20 mg for 1 g of the substrate, and the usage of pullulanase is from 0.1 mg to 10 mg for 1 g of the substrate.

The production method of the present invention may be used for the production of a hetero-oligosaccharide. When a hetero-oligosaccharide is produced, maltotriosyl transferase (preferably the present enzyme) is allowed to act on a saccharide in the presence of a receptor substrate. The saccharide is not particularly limited, and may be, for example, maltooligosaccharide, dextrin, or starch. Examples of the maltooligosaccharide include maltotetraose, maltopentaose, and maltohexaose. The receptor substrate is also not particularly limited, and may be, for example, monosaccharide or sugar alcohol. Examples of the monosaccharide and sugar alcohol include D-xylose, D-fructose, D-glucose, L-fucose, L-sorbose, D-mannose, D-sorbitol, D-galactose, N-acetyl glucosamine, L-arabinose, D-ribose, and L-rhamnose. The loading of the present enzyme is not particularly limited, and may be from 1 U to 400 U for 1 g of the substrate. The temperature during enzyme reaction is not particularly limited, and preferably from 30° C. to 70° C., thereby allowing the present enzyme to sufficiently act.

According to one embodiment, the production method of the present invention is carried out as a part of the production process of beer or a beer-like beverage. In typical cases, the preparation step (saccharification step) of beer or a beer-like beverage is carried out after the addition of maltotriosyl transferase, or maltotriosyl transferase is added to the saccharified solution obtained after the preparation step, thereby carrying out the enzyme reaction. In this manner, the maltotriosyl transferase is allowed to act on the saccharide formed by saccharification of the saccharide ingredient used in beer or a beer-like beverage. The beer or a beer-like beverage in the present invention is not limited to the beer under the Liquor Tax Act, and include general brews such as low-malt beer and general beer-taste beverages which are brewed using a beer yeast. Low alcohol beer, non-alcohol beer, and the third beer are also included in “beer or beer-like beverages”. Examples of the saccharide ingredient include malt, barley, wheat, rye, oat, corn, rice, kaoliang, potato, soybean, pea bean, chick-pea, and corn starch. As necessary, two or more ingredients are used in combination. In the production method according to this embodiment, beer or a beer-like beverage having a high indigestible saccharide (dietary fiber) content is obtained. In the production process of beer or a beer-like beverage, an enzyme such as glucoamylase, α-amylase, β-amylase, or pullulanase may be used in combination. These enzymes are used typically for the reduction of saccharides. These enzymes are added, for example, during the reaction of the maltotriosyl transferase.

The present invention also provides a composition containing the indigestible saccharide produced by the production method of the present invention. The composition of the present invention is not particularly limited as to its use, and is preferably used for medicine, quasi-drug, or food. More specifically, a preferred embodiment of the present invention is to provide a pharmaceutical composition, a quasi-drug composition, and a food composition containing the indigestible saccharide produced by the production method of the present invention.

The pharmaceutical composition and quasi-drug composition of the present invention may be formulated according to a common procedure. The formulation may contain other pharmaceutically acceptable other components (for example, carrier, excipient, disintegrating agent, buffer, emulsifying agent, suspending agent, soothing agent, stabilizer, preservative, antiseptic, and normal saline solution). Examples of the excipient include lactose, starch, sorbitol, D-mannitol, and white sugar. Examples of the disintegrating agent include starch, carboxymethyl cellulose, and calcium carbonate. Examples of the buffer include phosphates, citrates, and acetates. Examples of the emulsifying agent include gum arabic, sodium alginate, and tragacanth. Examples of the suspending agent include glyceryl monostearate, aluminum monostearate, methyl cellulose, carboxymethyl cellulose, hydroxymethyl cellulose, and sodium lauryl sulfate. Examples of the soothing agent include benzyl alcohol, chlorobutanol, and sorbitol. Examples of the stabilizer include propylene glycol and ascorbic acid. Examples of the preservative include phenol, benzalkonium chloride, benzyl alcohol, chlorobutanol, and methylparaben. Examples of the antiseptic include benzalkonium chloride, paraoxybenzoic acid, and chlorobutanol.

The dosage form of the formulation is also not particularly limited. The pharmaceutical composition or quasi-drug composition of the present invention may be provided in the form of, for example, pellets, powder, fine grains, granules, capsules, syrup, injection, external preparation, or suppository.

The pharmaceutical composition of the present invention contains an active ingredient in an amount necessary to achieve the expected therapeutical or prophylactic benefit (more specifically therapeutically effective amount). In addition, the quasi-drug composition of the present invention contains an active ingredient in an amount necessary to achieve the expected improving or prophylactic benefit. The amount of the active ingredient contained in the pharmaceutical composition or quasi-drug composition of the present invention is, for example, about 0.1% by weight to about 95% by weight depending on the dosage form or shape, thereby achieving a desired dose.

Examples of the “food composition” in the present invention include common food (grains, vegetable, meat, various processed food, confectionery, milk, refreshing beverage, alcohol beverage (for example, beer or beer-like beverage), food supplements (dietary supplements, nutrition drinks), food additives, pet food, and pet dietary supplements. Food supplements or food additives may be in the form of powder, granules, tablets, paste, or liquid. The form of a food composition makes it easy to routinely or continuously take the active ingredient of the present invention (indigestible saccharide). The food composition of the present invention preferably contains the active ingredient in an amount effective to achieve therapeutical or prophylactic benefit. The loading may be established in consideration of, for example, the disease state, health condition, age, sex, body weight of the subject.

### Use of Maltotriosyl Transferase 3: Method for Producing a Glycoside

A third aspect of the present invention is to provide a method for producing a glycoside as a use of maltotriosyl transferase. In the method for producing a glycoside according to the present invention, in the same manner as the first aspect, preferably the maltotriosyl transferase (the present enzyme) disclosed by the applicants in the preceding patent application (see Patent Document 2 for more detail) is used. More specifically, the method for producing a glycoside according to the present invention includes a step of allowing maltotriosyl transferase (preferably the present enzyme) to act on a donor substrate in the presence of a receptor substrate.

Various receptor substrates may be used herein (see the below-described Example). Specific examples of the receptor substrate include kojic acid, ascorbic acid, hydroquinone, glycerol, monoglyceride, and diglyceride. Two or more receptor substrates may be used in combination. The donor substrate is not particularly limited as long as the maltotriosyl transferase used can act thereon. Specific examples of the donor substrate include soluble starch, amylose, amylopectin, maltotetraose, maltopentaose, and maltohexaose. Two or more donor substrates may be used in combination. The loading of the present enzyme is not particularly limited, and may be from 1 U to 100 U for 1 g of the donor substrate. The temperature conditions during enzyme reaction are not particularly limited, and preferably from 30° C. to 70° C., thereby allowing the sufficient action of the present enzyme.

### Use of Maltotriosyl Transferase 4: Method for Producing a Dextrin Solution

The inventors found that a dextrin solution which was treated by maltotriosyl transferase does not become muddy after a long-term storage at low temperature. Based on this finding, as one of novel applications of maltotriosyl transferase, a production method of dextrin solution which is hard to retrograde is provided. The method comprises a step of allowing maltotriosyl transferase to act on dextrin in a solution.

Low DE dextrin solution tends to lose a gelatinization state. Long term storage makes the solution muddy. This problem can be solved by the present invention, namely by a treatment of maltotriosyl transferase. In other words, maltotriosyl transferase stabilizes the structure of dextrin and thereby makes it possible to store a low DE dextrin solution for a long term without retrograding. As a result, a low DE dextrin can be distributed in a form of solution which is easy to use, saves costs, etc. Low DE dextrin solution produced by the present invention can be applied to, for example, caramel, Japanese confectionery, beverage and frozen dessert.

The present invention also provides a composition containing the dextrin solution produced by the production method of the present invention. The composition of the present invention is not particularly limited as to its use, and is preferably used for food.

### EXAMPLES

### Measurement Method of Maltotriosyl Transferase Activity

The activity of the maltotriosyl transferase was measured as described below. More specifically, 0.5 mL of an enzyme solution was added to 2 mL of a 10 mmol/L MES buffer solution (pH 6.5) containing 1% maltotetraose (manufactured by Hayashibara Co., Ltd.), and allowed to stand at 40° C. for 60 minutes. After standing, the mixture was heated for 5 minutes in a boiling water bath, and cooled in running water. The glucose thus formed was quantified using Glucose CII-Test Wako (manufactured by Wako Pure Chemical Industries, Ltd.). Under these conditions, the amount of the enzyme forming 1 μmol of glucose in 2.5 mL of the reaction solution for 1 minute was set at 1 unit.

### Confirmation Method of Activity of Maltotriosyl Transferase

In addition to the above-described <Measurement method of maltotriosyl transferase activity>, the activity of maltotriosyl transferase was confirmed as described below. More specifically, 15 μL of 1.0 u/mL enzyme solution was added to 985 μL of 5 mmol/L acetic acid buffer solution (pH 6.0) containing 10.3 mmol/L maltotetraose (manufactured by Hayashibara Co., Ltd.), and allowed to stand at 50° C. for 1, 2, and 3 hours. After standing, the mixture was heated in a boiling water bath for 5 minutes, and then cooled in running water. The cooled reaction solution was desalted as needed using cationic and anionic resins, and the reaction solution was analyzed by HPLC. In the analysis, the HPLC apparatus was “Prominence UFLC” manufactured by Shimadzu Corporation, the column was “MCI GEL CK04S” manufactured by Mitsubishi Chemical Corporation, the eluent was water at a flow rate of 0.4 mL/minute, and the detector was a differential refractometer. The percentage of areas of the substrate and product thus obtained were converted into the molar quantity, and the consumption rate and production rate were calculated. For the purified maltotriosyl transferase, for example, the production rate ratio was heptaose:triose=about 92: about 8.

As described in the preceding patent application (Patent Document 2), maltotriosyl transferase from Geobacillus sp. APC9669 was successfully obtained. The method for obtaining the enzyme and characteristics of the enzyme are cited from Patent Document 2 and described below.

1. Production and Purification of Maltotriosyl Transferase from Geobacillus sp. APC9669

Geobacillus sp. APC9669 was cultured under shaking at 45° C. for 2 days using a liquid culture medium having the components shown in Table 1. The culture supernatant thus obtained was concentrated five-fold using a UF membrane (AIP-1013D, manufactured by Asahi Kasei Corporation), and then ammonium sulfate was added to give a 50% saturated concentration. The precipitate fraction was dissolved again in 20 mmol/L tris-hydrochloride buffer solution (pH 8.0) containing 5 mmol/L calcium chloride, and subsequently ammonium sulfate was added to give a final concentration of 0.5 mol/L. The precipitate thus formed was removed by centrifugation, the remaining solution was passed through an HiLoad 26/10 Phenyl Sepharose HP column (manufactured by GE Healthcare) which had been equilibrated with 20 mmol/L tris-hydrochloride buffer solution (pH 8.0) containing 0.5 mol/L ammonium sulfate and 5 mmol/L calcium chloride, and the adsorbed maltotriosyl transferase protein was eluted by linear concentration gradient of ammonium sulfate from 0.5 mol/L to 0 mol/L.

TABLE 1
Culture medium for producing maltotriosyl transferase

| | (w/v) |
|---|---|
| Yeast extract | 1.5% |
| Soybean peptone | 0.5% |
| Sodium chloride | 0.5% |
| Soluble starch | 0.4% |

The collected fraction containing active maltotriosyl transferase was concentrated using a UF membrane, and the buffer solution was replaced with a 20 mmol/L tris-hydrochloride buffer solution (pH 8.0) containing 5 mmol/L calcium chloride. The sample with a new buffer solution was passed through an HiLoad 26/10 Q Sepharose HP column (manufactured by GE Healthcare) which had been equilibrated with 20 mmol/L tris-hydrochloride buffer solution (pH 8.0) containing 5 mmol/L calcium chloride, and the adsorbed maltotriosyl transferase protein was eluted by linear concentration gradient of NaCl from 0 mol/L to 1 mol/L.

Furthermore, the collected fraction containing active maltotriosyl transferase was concentrated using a UF membrane, the buffer solution was replaced with a 50 mM phosphate buffer solution (pH 7.2) containing 0.15 M NaCl, and then the sample was passed through an HiLoad 26/60 Superdex 200 pg column (manufactured by GE Healthcare) which had been equilibrated with a 50 mM phosphate buffer solution (pH 7.2) containing 0.15 M NaCl, and eluted with the buffer. The fraction containing active maltotriosyl transferase was collected, desalted and concentrated using an ultrafiltration membrane, and thus a purified enzyme preparation was obtained. The purified enzyme thus obtained was subjected to the study of the following properties.

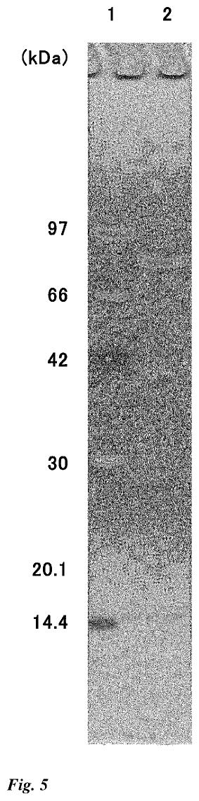

The results of the purification at each step are shown in Table 2. The specific activity at the final step was about 41 times in comparison with that of the crude enzyme. FIG. 5 shows the result of SDS-PAGE (CBB staining) on the samples at each step of purification using 10-20% gradient gel, indicating that the purified enzyme preparation (lane 2) is a single protein in SDS-PAGE.

TABLE 2

| | | | Specific | |
|---|---|---|---|---|
| | Total protein | Total activity | activity | Recovery |
| | (mg) | (U) | (u/mg) | (%) |
| Concentrate | 42 | 300 | 3.9 | 100 |
| Ammonium sulfate | 8.5 | 220 | 22.9 | 73 |
| fraction | | | | |
| Phenyl HP | 0.50 | 58 | 100 | 19 |
| Q HP | 0.37 | 53 | 139 | 18 |
| Superdex 200 | 0.12 | 19 | 158 | 6.3 |

### 2. Properties of Maltotriosyl Transferase

### (1) Optimal Reaction Temperature

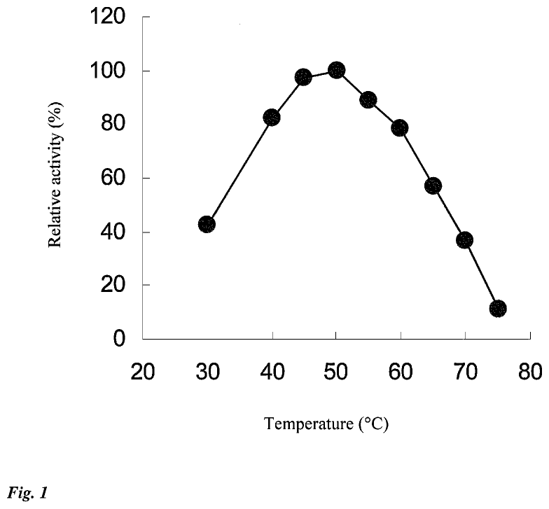

In the same manner as the above-described maltotriosyl transferase activity measurement method, the reaction was carried out at 30° C., 40° C., 45° C., 50° C., 55° C., 60° C., 65° C., 70° C., and 75° C. The results were expressed as relative activity, taking the value at the temperature which gave the maximum activity as 100%. The optimal reaction temperature was in the vicinity of 50° C. (FIG. 1).

### (2) Optimal Reaction pH

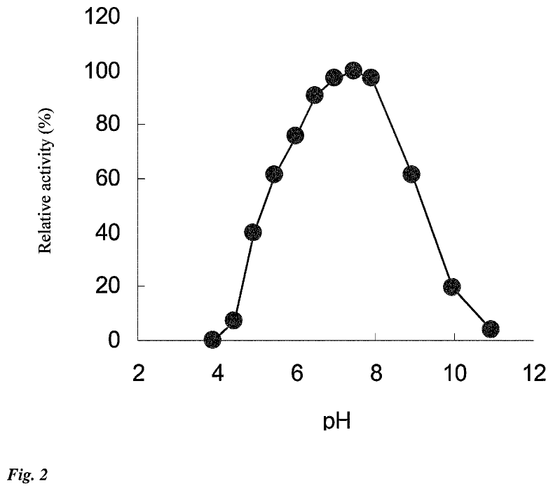

In the same manner as the above-described maltotriosyl transferase activity measurement method, the activity was measured under reaction conditions of 40° C. and 60 minutes in buffers (universal buffer solution pH 4.0, pH 4.5, pH 5.0, pH 5.5, pH 6.0, pH 6.5, pH 7.0, pH 7.5, pH 8.0, pH 9.0, pH 10.0, and pH 11.0). The results were expressed as relative activity, taking the value at the pH which gave the maximum activity as 100%. The optimal reaction pH was in the vicinity of about 7.5 (FIG. 2).

### (3) Thermostability

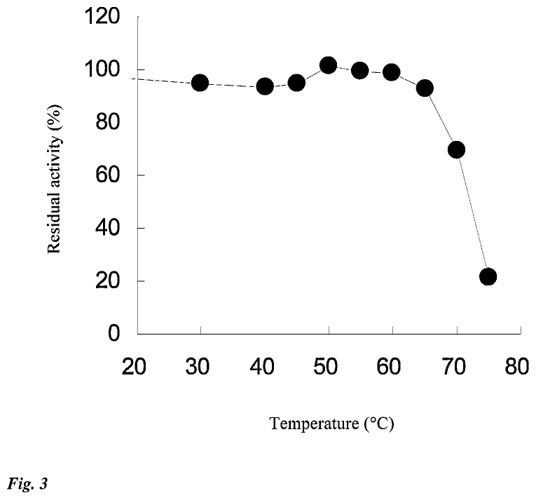

6 u/mL of enzyme solution was subjected to heat treatment in 10 mmol/L MES buffer solution (pH 6.5) for 30 minutes at 30° C., 40° C., 45° C., 50° C., 55° C., 60° C., 65° C., 70° C., and 75° C., and then the residual activity was measured by the above-described maltotriosyl transferase activity measurement method. The residual activity was expressed taking the activity untreated with heat as 100%. After heat treatment at 65° C. for 30 minutes, residual activity was 90% or more, and stable up to 65° C. (FIG. 3).

### (4) pH Stability

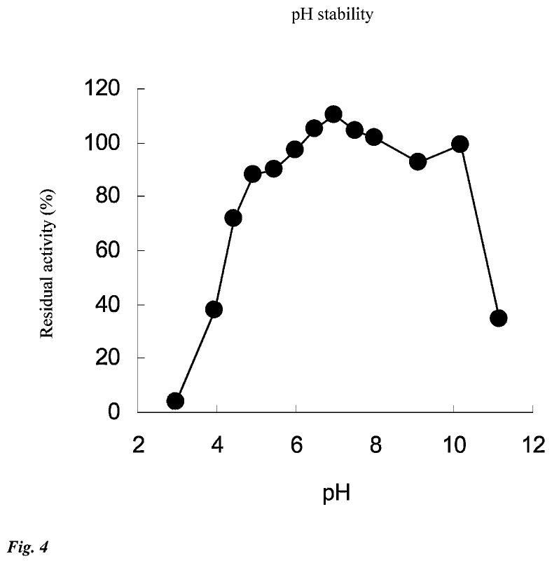

6 u/mL of enzyme solution was treated at 40° C. for 30 minutes in each buffer solution (universal buffer solution pH 3.0, pH 4.0, pH 4.5, pH 5.0, pH 5.5, pH 6.0, pH 6.5, pH 7.0, pH 7.5, pH 8.0, pH 9.0, pH 10.0, and pH 11.0), and the activity was measured by the above-described maltotriosyl transferase activity measurement method. In the range from pH 5.0 to pH 10.0, the residual activity was 85% or more, and stable from pH 5.0 to pH 10.0 (FIG. 4).

### (5) Molecular Weight Measurement by SDS-PAGE

SDS-PAGE was carried out in accordance with the method by Laemmli et al. The molecular weight marker used herein was Low Molecular Weight Calibration Kit for Electrophoresis (manufactured by GE Healthcare), which contained Phosphorylase b (97,000 Da), Albumin (66,000 Da), Ovalbumin (45,000 Da), Carbonic anhydrase (30,000 Da), Trypsin inhibitor (20,100 Da), and α-Lactalbumin (14,400 Da) as standard proteins. Electrophoresis was carried out for about 80 minutes at 20 mA/gel using a gradient gel (manufactured by Wako Pure Chemical Industries, Ltd.) having a gel concentration of 10-20%, and the molecular weight was determined; the molecular weight was about 83 kDa (FIG. 5).

### (6) Isoelectric Point

The isoelectric point of the present enzyme was about 4.5 as measured by isoelectric focusing using ampholine (600V, 4° C., a current was passed for 48 hours).

### (7) Substrate Specificity

The maltotriosyl transferase activity for different substrates was studied.

### a) Substrate Specificity for Maltooligosaccharide

Substrate specificity for maltooligosaccharide was studied as follows. The enzyme was added to each 10 mmol/L maltooligosaccharide to give a concentration of 0.002 u/mL, and allowed to stand at 50° C. for 1, 2, and 3 hours. After standing, the mixture was heated for 5 minutes in a boiling water bath, and then cooled in running water. The cooled reaction solution was desalted as needed using cationic and anionic resins, and the reaction solution was analyzed by HPLC. The HPLC apparatus was “Prominence UFLC” manufactured by Shimadzu Corporation, the column was “MCI GEL CK04S” manufactured by Mitsubishi Chemical Corporation, the eluent was water at a flow rate of 0.4 mL/minute, and the detector was a differential refractometer. The percentage of areas of the substrate and product thus obtained were converted into the molar quantity, and the consumption rate and production rate were calculated. The rate of reaction for each maltooligosaccharide was calculated as follows. The rate for maltotetraose was the sum of the formation rate of heptaose and triose. The rate for maltopentaose was the sum of the production rate of octaose and triose. The rate for maltohexaose was calculated by halving the difference between the production rates of triose and nonose, and adding the value to the production rate of nonose.

TABLE 3

| Substrate | Relative rate (%) |
|---|---|
| Maltose | 0 |
| Maltotriose | 0 |
| Maltotetraose | 83 |
| Maltopentaose | 100 |
| Maltohexaose | 89 |

For maltose and maltotriose, no reaction product was found. Sufficient action was observed on maltotetraose, maltopentaose, and maltohexaose.

### b) Substrate Specificity for Polysaccharide

For cyclodextrin, soluble starch, amylose, and amylopectin, the substrate specificity was studied by the following method. The enzyme was added at 0.002 u/mL with reference to 10 mmol/L of each maltooligosaccharide, and 0.1 u/mL of enzyme was allowed to stand at 50° C. for 0, 1, 2, and 3 hours. After standing, the mixture was heated for 5 minutes in a boiling water bath, and cooled in running water. To 200 μL of the solution, Rhizopus-derived glucoamylase (manufactured by Wako Pure Chemical Industries, Ltd.) was added at 0.03 mg for 1.0 unit, and allowed to stand at 50° C. overnight. After standing, the mixture was heated in a boiling water bath for 5 minutes, and then cooled in running water. The cooled reaction solution was desalted as needed using cationic and anionic resins, and the reaction solution was analyzed by HPLC. The HPLC apparatus was “Prominence UFLC” manufactured by Shimadzu Corporation, the column was “MCI GEL CK04S” manufactured by Mitsubishi Chemical Corporation, the eluent was water at a flow rate of 0.4 mL/minute, and the detector was a differential refractometer. In the comparison between the groups treated and untreated with the enzyme (maltotriosyl transferase), when the peaks of triose or higher saccharides increased with time, the product was judged as present (+), and when no increase was found, the product was judge as absent (−).

TABLE 4

| Substrate | Presence or absence of product |
|---|---|
| α-cyclodextrin | − |
| β-cyclodextrin | − |
| γ-cyclodextrin | − |
| Amylose | + |
| Amylopectin | + |
| Soluble starch | + |

For cyclodextrin, no reaction product was found. For soluble starch, amylose, and amylopectin, peaks of triose or higher saccharides increased with time. These polysaccharides were found to work as substrates. Since glucoamylase hydrolyzes α-1,4 and α-1,6 bonds, it was found that transglycosylation products were formed for other bonding patterns.

### (8) Influence of Substrate Concentration on Enzyme Reaction Product

The influence of the substrate concentration on the enzyme reaction product was studied using maltotetraose as the substrate. The enzyme was added in such a manner that the residual amount of maltotetraose after reaction for 3 hours was 85% or more with reference to 0.67, 1.0, 3.0, 10, 30, and 70% (w/v) maltotetraose, and allowed to stand at 50° C. for 1, 2, and 3 hours. After standing, the mixture was heated in a boiling water bath for 5 minutes, and cooled in running water. The cooled reaction solution was desalted as needed using cationic and anionic resins, and the reaction solution was analyzed by HPLC. The HPLC apparatus was “Prominence UFLC” manufactured by Shimadzu Corporation, the column was “MCI GEL CK04S” manufactured by Mitsubishi Chemical Corporation, the eluent was water at a flow rate of 0.4 mL/minute, and the detector was a differential refractometer. The percentage of areas of the substrate and product thus obtained were converted into the molar quantity, and the production rate was calculated. As a result of this, the rate of transglucosylation was 90% or more at all the substrate concentrations (0.67 to 70% (w/v)).

TABLE 5

| | Reaction formation rate (molar ratio) | |
|---|---|---|
| Substrate concentration | Transfer product | Decomposition product |
| (% (w/v)) | (heptaose) | (triose) |
| 0.67 | 92% | 8% |
| 1.0 | 96% | 4% |
| 3.0 | 100% | 0% |
| 10 | 100% | 0% |
| 30 | 100% | 0% |
| 70 | 100% | 0% |

### 3. Production of Rice Cake

### 3-1. Production of Rice Cake

200 g of water was added to 250 g of rice flour and an enzyme (maltotriosyl transferase, BIOZYME M5 (derived from soybean, manufactured by Amano Enzyme Inc.) or α-glucosidase “AMANO” SD (manufactured by Amano Enzyme Inc.), and the mixture was steamed for 15 minutes with water vapor. The loading of maltotriosyl transferase was 8 u or 12 u/g (substrate). The loading of BIOZYME M5 was 1.2 mg/g (substrate), and the loading of α-glucosidase “AMANO” SD was 1.2 mg/g (substrate).

Subsequently, the steamed object was stirred in a mixer (KITCHEN AID KSM5 manufactured by FMI Corporation), molded in a plastic petri dish, allowed to cool, and stored at 5° C. After storing for one day or two days, the center of the rice cake was die-cut into a column having a diameter of 25 mm. In order to measure the hardness of rice cake, the maximum load when the rice cake was compressed 50% at a compression speed of 60 mm/minute was measured using a rheometer (COMPAC-100II manufactured by Sun Scientific Co., Ltd.). The hardness of the rice cake of different test groups was compared, taking the hardness of the rice cake after storing for 2 hours as 100%.

In the control test group (enzyme was added after steaming), firstly, 250 g of rice flour and 200 g of water were added, and then the mixture was steamed with water vapor for 15 minutes. Subsequently, the steamed object was stirred in a mixer (KITCHEN AID KSM5, manufactured by FMI Corporation), and 300 mg of BIOZYME M5 (derived from soybean, manufactured by Amano Enzyme Inc.) was added under stirring when the temperature of the dough reached about 65° C. The mixture was molded in a plastic petri dish, allowed to cool, and stored at 5° C.

As a result of comparative evaluation, the group containing 12 u/g of maltotriosyl transferase (substrate) showed softness keeping effect equivalent to the control test group. In addition, the group showed no excessive stickiness of the rice cake, and maintained favorable physical properties. Accordingly, the maltotriosyl transferase may be added before steaming, and may be mixed with a premix powder, different from prior art soybean-derived β-amylase and α-glucosidase (transglucosidase).

TABLE 6

| | | | Hardness (%) | | |
|---|---|---|---|---|---|
| Loading of enzyme (/g-substrate) | | | After | After | |
| G3 | BIOZYME | α- | storage for | storage for | |
| transferase | M5 | glucosidase | 1 day | 2 days | Remark |
| 0 | 0 | 0 | x | x | |
| 8 u | 0 | 0 | 115 | 137 | |
| 12 u | 0 | 0 | 104 | 75 | |
| 0 | 1.2 mg | 0 | 202 | 380 | |
| 0 | 0 | 1.2 mg | x | x | |
| 0 | 1.2 mg | 0 | 101 | 69 | Control test |
| | | | | | group (after |
| | | | | | steaming) |

G3 transferase: maltotriosyl transferase
X: Measurement was impossible because too hard

### 3-2. Production of Rice Cake (With the Addition of pH Controlling Agent)

200 g of 50 mmol/L acetic acid buffer solution (pH 5.3) was mixed with 250 g of rice flour and an enzyme (maltotriosyl transferase, BIOZYME M5 derived from soybean, manufactured by Amano Enzyme Inc., or α-glucosidase “AMANO” SD, manufactured by Amano Enzyme Inc.), and the mixture was steamed with water vapor for 15 minutes. The loading of the maltotriosyl transferase was 8 u or 12 u/g (substrate). The loading of BIOZYME M5 was 1.2 mg/g (substrate), and the loading of α-glucosidase “AMANO” SD was 1.2 mg/g (substrate).

Subsequently, the steamed object was stirred in a mixer (KITCHEN AID KSM5, manufactured by FMI Corporation), molded in a plastic petri dish, allowed to cool, and stored at 5° C. After storing for one day or two days, the center of the rice cake was die-cut into a column having a diameter of 25 mm. In order to measure the hardness of rice cake, the maximum load when the rice cake was compressed 50% at a compression speed of 60 mm/minute was measured using a rheometer (COMPAC-100II manufactured by Sun Scientific Co., Ltd.). The hardness of the rice cake of different test groups was compared, taking the hardness of the rice cake after storing for 2 hours as 100%.

In the control test group (enzyme was added after steaming), firstly, 200 g of 50 mmol/L acetic acid buffer solution (pH 5.3) was added to 250 g of rice flour, and steamed with water vapor for 15 minutes. Subsequently, the steamed object was stirred in a mixer (KITCHEN AID KSM5, manufactured by FMI Corporation), and 300 mg of BIOZYME M5 (derived from soybean, manufactured by Amano Enzyme Inc.) was added under stirring when the temperature of the dough reached about 65° C. The mixture was molded in a plastic petri dish, allowed to cool, and stored at 5° C.

The result of the comparative evaluation, the enzyme-added groups containing 8 and 12 u/g (substrate) of maltotriosyl transferase showed the same softness keeping effect as the control test group. In addition, excessive stickiness of the rice cake was not detected, and favorable physical properties were maintained. Accordingly, the maltotriosyl transferase may be added before steaming, and may be mixed with a premix powder even in the presence of a pH controlling agent, different from prior art soybean-derived β-amylase and α-glucosidase (transglucosidase)

TABLE 7

| | | | Hardness (%) | | |
|---|---|---|---|---|---|
| Loading of enzyme (/g-substrate) | | | After | After | |
| G3 | BIOZYME | α- | storage for | storage for | |
| transferase | M5 | glucosidase | 1 day | 2 days | Remark |
| 0 | 0 | 0 | x | x | |
| 8 u | 0 | 0 | 123 | 88 | |
| 12 u | 0 | 0 | 107 | 76 | |
| 0 | 1.2 mg | 0 | 289 | 604 | |
| 0 | 0 | 1.2 mg | X | X | |
| 0 | 1.2 mg | 0 | 100 | 56 | Control test |
| | | | | | group (after |
| | | | | | steaming) |

G3 transferase: maltotriosyl transferase
X: Measurement was impossible because too hard

### 4. Production of Indigestible Saccharide

### (1) Measurement Method of Indigestible Component Content

The measurement was carried out by the improved method of “Determination of Indigestible Components” (Denpun Kagaku (Journal of the Japanese Society of Starch Science), vol. 37, No. 2, p. 107, 1990). Firstly, 1 mL of the sample was adjusted to 15 mL using 50 mmol/L phosphate buffer solution (pH 6.0), 30 μL of α-amylase (equivalent to TERMAMYL 120L manufactured by Wako Pure Chemical Industries, Ltd.) was added, and the mixture was allowed to react at 95° C. for 30 minutes. After cooling, the pH was readjusted to 4.5, 30 μL of amyloglucosidase (manufactured by Sigma-Aldrich Co. LLC) was added, and the mixture was allowed to react at 95° C. for 30 minutes. The reaction was stopped by boiling in a boiling water bath for 5 minutes. The volume of the reaction solution was adjusted to 30 mL, and the indigestible component content was calculated by the pyranose-oxidase method using the following equation.

Indigestible component content (%)=100−formed amount of glucose (%)×0.9

### (2) Preparation of Indigestible Saccharide by Maltotriosyl Transferase 1

An indigestible saccharide was produced by adding maltotriosyl transferase to dextrin. Maltotriosyl transferase and a starch hydrolysate (PINEDEX #100 (manufactured by Matsutani Chemical Industry Co,. Ltd., the final concentration 30% (w/v)) were added to 10 mmol/L MES buffer solution (pH 6.0), and the solution amount was adjusted to 20 mL. In order to study the effect of the combination of the maltotriosyl transferase with the other enzyme, the test groups using the combination of the maltotriosyl transferase with cyclodextrin glucanotransferase (TORUZYME 3.0 L manufactured by Novozymes) and/or pullulanase (KLEISTASE PL45 manufactured by Amano Enzyme Inc.) were also prepared.

After reaction at 60° C. for 66 hours, the reaction was stopped by boiling in a boiling water bath for 10 minutes. The cooled reaction solution was subjected to the above-described “(1) Measurement method of indigestible component content”.

TABLE 8

| | | | Indigestible |
|---|---|---|---|
| Loading of enzyme (/g-substrate) | | | component content |
| G3 transferase | CGTase | Pullulanase | (%) |
| 20 u | 0 | 0 | 49.5 |
| 20 u | 3 mg | 0 | 51.9 |
| 20 u | 10 mg | 0 | 57.9 |
| 20 u | 0.3 mg | 1 mg | 52.1 |
| 20 u | 3 mg | 1 mg | 61.6 |
| 20 u | 10 mg | 1 mg | 63.5 |

G3 transferase: maltotriosyl transferase
CGTase: cyclodextrin glucanotransferase

Table 8 indicates that the indigestible component contents in the products were 49.5% for the single addition of maltotriosyl transferase (20 u/g (substrate)), 57.9% for the combined addition of maltotriosyl transferase (20 u/g (substrate)) and cyclodextrin glucanotransferase (10 mg/g (substrate)), and 63.5% for the combined addition of three enzymes, or maltotriosyl transferase (20 u/g (substrate)), cyclodextrin glucanotransferase (10 mg/g (substrate)), and pullulanase (1 mg/g (substrate)).

As described above, it was indicated that the maltotriosyl transferase can produce an indigestible saccharide even by the reaction at a high temperature. The reaction at a high temperature is effective for reducing the risk of microorganism contamination, and is particularly demanded for the production on industrial scale. In addition, it was found that the combined use with cyclodextrin glucanotransferase or pullulanase can increase the indigestible saccharide content. In particular, the combined use of three enzymes composed of maltotriosyl transferase, cyclodextrin glucanotransferase, and pullulanase, increased the indigestible saccharide content by 10% or more in comparison with the single use of maltotriosyl transferase.

### 5. Dietary Fiber Increasing Effect in Beer or Beer-Like Beverage

The saccharification reaction used an automatic saccharification tank LB-8 (manufactured by LOCHNER Ltd.). 320 g of 1.2 mmol/L calcium sulfate aqueous solution was charged into the saccharification bath, the temperature was increased to 50° C. under stirring, and then 8 g of malt, 72 g of ground barley, and maltotriosyl transferase were added. The loading of the maltotriosyl transferase was 0 u, 96 u or 480 u. After adding the ingredients and the enzyme, the mixture was treated at 50° C. for 1 hour. Furthermore, the temperature was increased, kept at 65° C. for 1 hour, and then increased again and kept at 76° C. for 10 minutes. The saccharified solution thus obtained was filtered through filter paper to obtain a filtrate. The content of the water-soluble dietary fiber in the filtrate was determined by the enzyme-HPLC method. As a result of this, the water-soluble dietary fiber content was 1.7% (w/v) for the enzyme-free group, 2.6% (w/v) for the enzyme 96 u-added group, and 3.2% (w/v) for the 480 u-added group. Dietary fiber will not be utilized by the beer yeast during the subsequent fermentation step. Therefore, the increase of dietary fiber during the saccharification step results in the increase of dietary fiber in the beer. The addition of maltotriosyl transferase increased the dietary fiber in the wort, irrespective of the malt content in the ingredients.

### 6. Production of Noodle

90 mL of tap water containing 300 u of maltotriosyl transferase (the enzyme-free group is tap water alone) was added to 100 g of rice flour (“POWDER RICE D” manufactured by Niigata Seifun K.K.). The temperature was kept at about 66° C. for 10 minutes, the thoroughly kneaded mixture was spread by a paste machine (“Pasta Machine IMPERIA SP-150” manufactured by Imperia Company), and cut into noodles of 7 mm width. The noodles were steamed for 15 minutes in a steamer to obtain a noodle sample. The sample was tasted after a lapse of 2 minutes and after storage at 5° C. for 60 minutes; toughness was higher, and softness after storage for 60 minutes was maintained in comparison with the enzyme-free group. Therefore, it was confirmed that the taste of the noodles is maintained by the addition of maltotriosyl transferase.

### 7. Production of Hetero-Oligosaccharide

Maltotetraose as the donor substrate and a receptor substrate (D-xylose, D-fructose, D-glucose, L-fucose, L-sorbose, D-mannose, D-sorbitol, D-galactose, N-acetylglucosamine, L-arabinose, D-ribose, or L-rhamnose) were dissolved in 10mmol/L MES buffer solution (pH 6.5) to give a final concentration of 3.0% (w/v) or 10.0% (w/v). 1 u of maltotriosyl transferase was added to 1 mL of the substrate solution, and the mixture was allowed to stand and react in a water bath at 50° C. for 24 hours. Subsequently, the reaction was stopped by boiling for 10 minutes. The reaction solution was subjected to HPLC analysis. In the HPLC analysis, the column was CK04S manufactured by Mitsubishi Chemical Corporation, the column temperature was 80° C., the mobile phase was ultrapure water at a flow rate of 0.4 mL/min, and the detector was a differential refractometer. The formation of the product was calculated, taking the formation of the receptor substrate before reaction as 100%. As shown in Table 9, various hetero-oligosaccharides were produced using various receptor substrates in the presence of maltotriosyl transferase. These hetero-oligosaccharides likely have various functions of commercially available functional oligosaccharides, such as anticarious action and intestine regulation action. Examples of the donor substrate other than maltotetraose include dextrin and starch.

TABLE 9

| Receptor substrate | Formation (%) |
|---|---|
| D-xylose | 26.3 |
| D-fructose | 17.0 |
| D-glucose | 15.9 |
| L-fucose | 12.9 |
| L-sorbose | 12.5 |
| D-mannose | 11.3 |
| D-sorbitol | 9.2 |
| D-galactose | 8.2 |
| N-acetylglucosamine | 8.1 |
| L-arabinose | 7.8 |
| D-ribose | 7.7 |
| L-rhamnose | 3.3 |

### 8. Production of Glycoside 1

Amylose EX-1 as the donor substrate and a receptor substrate (kojic acid, ascorbic acid, or hydroquinone) were dissolved in 5 mmol/L MES buffer solution (pH 6.0) to give a final concentration of 2.5% (w/v). 1 u of maltotriosyl transferase was added to 1 mL of the substrate solution, and allowed to react in a water bath at 50° C. for 24 hours. Subsequently, the reaction was stopped by boiling for 10 minutes. The reaction solution was subjected to HPLC analysis. In the HPLC analysis, the column was TSK gel ODS-100V 3 um (4.6 mm×7.5 cm; manufactured by Tosoh Corporation), the column temperature was 50° C., the flow rate was 1.0 mL/min, and the detector was a UV detector. The mobile phase was composed of a solution A of 0.1% phosphoric acid and a solution B of methanol: ultrapure water=7:3, and elution was carried out in the pattern of 0 minutes (B: 0%)→4 minutes (B: 0%)→8 minutes (B: 100%)→10 minutes (B: 100%→B: 0%)→15 minutes (B: 0%). The formation of the product was calculated, taking the molar amount of the receptor substrate before reaction as 100%. Various glycosides were produced by the use of maltotriosyl transferase. Sugar bonding by maltotriosyl transferase allows the production of a glycoside having improved functions and properties, such as improved solubility and stability of the substance.

TABLE 10

| Receptor substrate | Formation (%) |
|---|---|
| Kojic acid | 6.1 |
| Ascorbic acid | 1.9 |
| Hydroquinone | 1.5 |

### 9. Production of Glycoside 2

Maltotetraose as the donor substrate and glycerol as the receptor substrate were dissolved in 10 mmol/L MES buffer solution (pH 6.5) to give a final concentration of 3.0% (w/v) or 10.0% (w/v). 1 u of maltotriosyl transferase was added to 1 mL of the substrate solution, and allowed to stand and react in a water bath at 50° C. for 24 hours. Subsequently, the reaction was stopped by boiling for 10 minutes. The reaction solution was subjected to HPLC analysis. In the HPLC analysis, the column was CK04S (manufactured by Mitsubishi Chemical Corporation), the column temperature was 80° C., the mobile phase was ultrapure water at a flow rate of 0.4 mL/min, and the detector was a differential refractometer. The formation of the product was calculated, taking the amount of the receptor substrate before reaction as 100%. Through the use of maltotriosyl transferase, a glycerol glycoside was produced in a molar amount of 7.3% with reference to the receptor substrate. The glycerol glycoside is a non-reducing glycoside contained in sake mash, mirin (Japanese sweet rice wine for cooking), and miso, and is said to impart sharp sweetness and richness to Japanese sake. In addition, the degree of sweetness of the glycoside is half that of sugar, and the glycoside can be added to food and beverages as a sweetener which has a low browning potential, low Maillard reactivity, high thermal stability, anticarious effect, indigestibility, low hygroscopicity, and high moisture retentivity.

### 10. Anti-Retrograding Effect of Maltotriosyl Transferase on Dextrin Solution

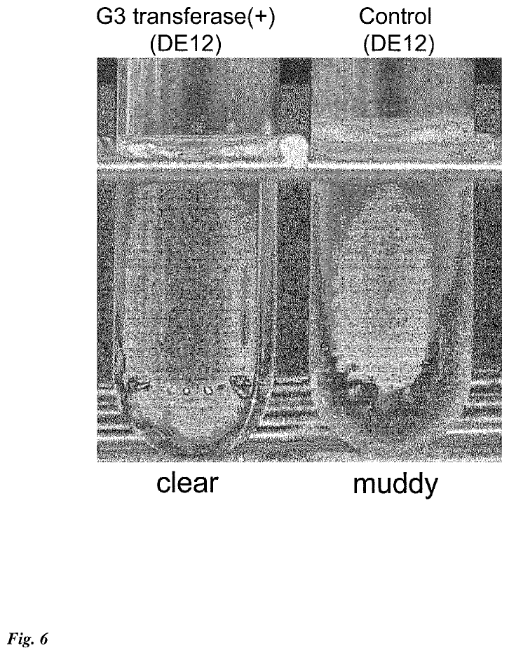

Dextrin (DE8) (PINEDEX #1 manufactured by Matsutani Chemical Industry Co. Ltd.) was dissolved in 10 mM MES Buffer (pH6.0) to give a final concentration of 30% (w/w). G3 transferase (20 u/g-dry substrate) was added to the substrate solution and allowed to react at 60° C. for 42 hours. The reaction was stopped by boiling for 10 minutes. Then, the reaction solution containing dextrin (DE12), which was a reaction product, was stored at 5° C. for two months (Sample). As a control, the solution containing Dextrin (DE12) (PINEDEX #2 manufactured by Matsutani Chemical Industry Co. Ltd.) was stored under the same condition. As shown in FIG. 6, the sample solution is clear whereas the control solution is muddy.

### INDUSTRIAL APPLICABILITY

The present invention provides a method for producing rice cakes using maltotriosyl transferase. The production method of the present invention can be used for the production of various rice cakes. The present invention provides a method for producing an indigestible saccharide. The indigestible saccharide obtained by the production method of the present invention is expected to find uses in the food and drug fields.

The present invention will not be limited to the above-described embodiments and examples of the present invention. The present invention includes various modifications which can be readily made by those skilled in the art without departing from the claims.

The entire contents of literatures, unexamined patent publications, and patent publications specified herein are incorporated herein by reference.

1-20. (canceled)

21. A method for producing a saccharide, comprising:
preparing an enzyme which acts on polysaccharides and oligosaccharides having α-1,4 glucoside bonds to transfer maltotriose units to saccharides;
producing an indigestible saccharide by reacting saccharides with maltotriosyl transferase; and
collecting the indigestible saccharide.

22. An enzyme preparation comprising an enzyme which acts on polysaccharides and oligosaccharides having α-1,4 glucoside bonds to transfer maltotriose units to saccharides.

23. Use of an enzyme which acts on polysaccharides and oligosaccharides having α-1,4 glucoside bonds to transfer maltotriose units to saccharides to produce an indigestible saccharide.

24. The method according to claim 21, wherein the step is carried out using one or more enzymes selected from the group consisting of cyclodextrin glucanotransferase, α-amylase (only those having transglycosylation activity), pullulanase, and isoamylase in combination with the maltotoriosyl transferase.

25. The method according to claim 21, wherein the reaction temperature during the step is from 30° C. to 70° C.

26. The method according to claim 21, wherein the polysaccharide or oligosaccharide having α-1,4 glucoside bonds is formed by saccharification of a saccharide ingredient used for producing beer or a beer-like beverage.

27. The method according to claim 21, wherein the maltotriosyl transferase is an enzyme derived from a microorganism.

28. The method according to claim 21, wherein the maltotriosyl transferase is an enzyme derived from Geobacillus species.

29. The method according to claim 21, wherein the maltotriosyl transferase has the following enzymatic properties:
(1) action: the enzyme acts on polysaccharides and oligosaccharides having α-1,4 glucoside bonds to transfer maltotriose units to saccharides;
(2) substrate specificity: the enzyme acts on soluble starch, amylose, amylopectin, maltotetraose, maltopentaose, and maltohexaose, but does not act on α-cyclodextrin, β-cyclodextrin, γ-cyclodextrin, maltotriose, and maltose; and
(3) molecular weight: about 83,000 Daltons by SDS-PAGE.

30. A composition containing an indigestible saccharide produced by the production method according to claim 21.

31. A method for producing a dextrin solution, comprising:
allowing maltotriosyl transferase having the SEQ ID NO: 3 to act on dextrin in a solution;
maintaining the contact of the dextrin with the maltotriosyl transferase under a reaction temperature above 30° C.; and
producing an indigestible saccharide by reacting dextrin with maltotriosyl transferase.

32. A dextrin solution produced by the production method according to claim 31.

33. A composition comprising the dextrin solution according to claim 32.
